(19) United States
(12) Patent Application Publication
(10) Pub. No.: US 2023/0303724 A1
(43) Pub. Date: Sep. 28, 2023

(54) HETEROPOLY OLIGOSACCHARIDE AND APPLICATION THEREOF IN IMPROVING PLANT DISEASE RESISTANCE

(71) Applicant: CHANGZHOU ENDELAN CHEMICALS IMP. & EXP. CO., LTD., Changzhou (CN)

(72) Inventors: Jianfa ZHANG, Nanjing (CN); Renjie FU, Nanjing (CN); Jing LI, Nanjing (CN); Rui CHENG, Nanjing (CN)

(21) Appl. No.: 18/297,269

(22) Filed: Apr. 7, 2023

Related U.S. Application Data
(63) Continuation of application No. PCT/CN2021/136586, filed on Dec. 8, 2021.

(30) Foreign Application Priority Data
Dec. 9, 2020 (CN) 202011427267.0

(51) Int. Cl. C08B 37/00; C12N 1/20; A01N 43/90

(52) U.S. Cl. CPC C08B 37/006; A01N 43/90; C08B 37/0003; C12N 1/20; C12R 2001/41

(57) ABSTRACT

The present disclosure provides a heteropoly oligosaccharide and an application thereof in improving plant disease resistance. The heteropoly oligosaccharide includes seven D-glucose residues and one D-galactose residue. Each heteropoly oligosaccharide molecule comprises R₁ and R₂, R₁ being H or a monomolecular pyruvate group, R₂ being H or a monomolecular succinyl group, and the structure thereof is represented as follows:

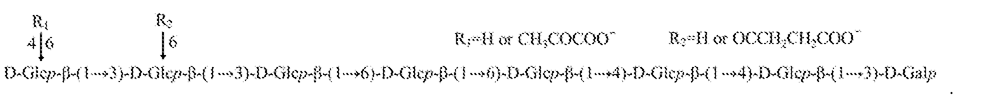

### CROSS-REFERENCES TO RELATED APPLICATIONS

This application is a continuation application of PCT Patent Application No. PCT/CN2021/136586, filed on Dec. 8, 2021, which claims priority to Chinese Patent Application No. 202011427267.0, filed on Dec. 9, 2020, the entire contents of both of which are incorporated herein by reference.

### FIELD OF THE DISCLOSURE

The present disclosure belongs to the field of plant disease resistance inducers, and relates to a heteropoly oligosaccharide and an application thereof for improving plant disease resistance.

### BACKGROUND

Effective autoimmune systems have been formed in plants in response to invasions of pathogenic bacteria through long-term evolution and development. Plant resistance inducers can stimulate immune systems of plants to resist, prevent and treat diseases. With respect to interactions between plants and pathogens, on one hand, plants secrete β-1,3 glucanase, chitosanase and chitinase to directly hydrolyze cell walls of pathogenic bacteria and inhibit their growth; on the other hand, pathogenic bacteria secrete polygalacturonase and pectinase to degrade cell walls of plants. Oligosaccharide fragments produced through the mutual hydrolyses can stimulate plants to produce pathogenesis-related proteins, phytoalexins, etc., thereby enhancing plant disease resistance. This is the basic principle of oligosaccharides as plant disease resistance inducers.

Low-DP β-1,3-glucooligosaccharides prepared from laminarin have been proved to have a favorable disease prevention effect on various plants, and relevant products have been marketed. However, oligosaccharides produced from laminarin have a low DP and are not uniform due to limitations of sources of raw materials and the hydrolysis process, making it difficult to give full play to their functions. Therefore, there is an urgent need to find an ideal alternative raw material to efficiently prepare oligosaccharides with a high DP and a uniform structure, and to explore the plant induction mechanism of new oligosaccharides.

The disclosed methods and applications are directed to solve one or more problems set forth above and other problems.

### SUMMARY

One aspect of the present disclosure provides a heteropoly oligosaccharide. The heteropoly oligosaccharide contains seven D-glucose residues and one D-galactose residue. Each heteropoly oligosaccharide molecule comprises R₁ and R₂, R₁ being H or a monomolecular pyruvate group, R₂ being H or a monomolecular succinyl group, and the structure thereof is represented as follows:

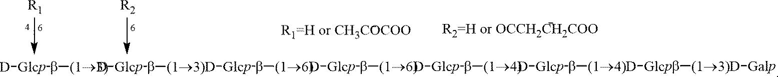

Another aspect of the present disclosure provides a preparation method of the above-described heteropoly oligosaccharide. The heteropoly oligosaccharide of the present disclosure can be prepared through enzymolysis of an exopolysaccharide Riclin under an action of β-glucanase, the exopolysaccharide Riclin being produced from Agrobacterium sp. ZCC3656 (CCTCC No.: M 2018797). Here, CCTCC is abbreviation of China Center for Type Culture Collection. The Agrobacterium sp. ZCC3656 has been disclosed in Chinese Patent Application No. 201811493131.2.

Another aspect of the present disclosure provides an application of the heteropoly oligosaccharide in improving plant disease resistance.

Another aspect of the present disclosure provides a plant disease resistance inducer with an active ingredient containing the heteropoly oligosaccharide.

Other aspects of the present disclosure can be understood by those skilled in the art in light of the description, the claims, and the drawings of the present disclosure.

### BRIEF DESCRIPTION OF THE DRAWINGS

The embodiments are shown and clarified with reference to the drawings. These drawings serve to clarify the basic principle, so that only aspects necessary for understanding the basic principle are shown. The drawings are not to scale. In the drawings, the same reference numerals indicate similar features.

FIG. 1 is a diagram showing changes of H₂O₂ concentration in tobacco leaves induced by a heteropoly oligosaccharide.

FIG. 2 shows H₂O₂ concentrations in cells of wheat leaves induced by the heteropoly oligosaccharide as observed through H₂DCF-DA fluorescence detection, where CK is a control group sprayed with water, 50 ppm means that the concentration of the heteropoly oligosaccharide is 50 mg/L, and 500 ppm means that the concentration of the heteropoly oligosaccharide is 500 mg/L.

FIG. 3 shows activities of resistance-related enzymes in tomato leaves induced by the heteropoly oligosaccharide, where a shows activity of glucanase; b shows activity of chitinase, and c shows activity of phenylalanine ammonia lyase.

FIG. 4 shows effects of the heteropoly oligosaccharide on potato leaves against Phytophthora infestans infection, where CK is a control group sprayed with water, 50 ppm means that the concentration of the heteropoly oligosaccharide is 50 mg/L, and 500 ppm means that the concentration of the heteropoly oligosaccharide is 500 mg/L.

FIG. 5 shows hydrogen nuclear magnetic resonance (NMR) spectrum of the heteropoly oligosaccharide consistent with some embodiments of the present disclosure.

FIG. 6 shows carbon-13 NMR spectrum of the heteropoly oligosaccharide consistent with some embodiments of the present disclosure.

Other features, characteristics, advantages and benefits of the present disclosure will become more apparent through the following detailed description in conjunction with the accompanying drawings.

### DETAILED DESCRIPTION

In the following detailed description of the preferred embodiments, reference will be made to the accompanying drawings constituting a part of the present disclosure. The drawings show specific embodiments capable of implementing the present disclosure by way of example. The exemplary embodiments are not intended to be exhaustive of all embodiments according to the present disclosure. It can be understood that other embodiments can be utilized, and structural or logical modifications can also be made without departing from the scope of the present disclosure. Therefore, the following detailed description is not restrictive, and the scope of the present disclosure is defined by the appended claims.

At present, research on the mechanism of oligosaccharides has developed from general observation to molecular and cellular levels. Many studies reveal that oligosaccharides play a role of elicitors generally in the following manner. Mutual recognition of oligosaccharides and receptors on cell membranes causes changes in receptor conformation and produces transmembrane signals; through a series of signal transduction, amplification and integration, expression of defense genes is regulated, secondary metabolites are accumulated, and plants are induced to produce autoimmune resistance to infection of pathogenic substances.

Oligosaccharide elicitors are derived from natural products and have the following functional characteristics. (1) They are ecologically-friendly. (2) They are effective against pathogenic bacteria that are ineffectively prevented and treated by conventional methods, especially those resistant to chemical pesticides. (3) They act on plants rather than directly on pathogenic organisms, which avoids adverse effects on non-pathogenic organisms. (4) They protect plants from various biotic stresses of microorganisms, insects, nematodes, etc. (5) They can be combined with other prevention and treatment methods acting in different ways to expand the scope of prevention and treatment. (6) They activate various genes for systemic resistance, some of which may protect plants from high and low temperatures, drought and ultraviolet stresses.

Oligosaccharide elicitors mainly include the following types. (1) Chitosan oligosaccharides: oligosaccharide products with a degree of polymerization (DP) between 2 and 20 obtained through the degradation of chitosan by means of a special biological enzyme technology; can induce plant disease resistance, elicit immunity against and kill various fungi, bacteria and viruses; have favorable prevention and treatment effects on cotton verticillium wilt, rice blast, tomato late blight and other diseases; and can be developed into biological pesticides, growth regulators, fertilizers, etc. (2) Chitin oligosaccharides: degradation products of chitin, are recognized as a general term of saccharides composed of 2-10 N-acetylglucosamines linked by glycosidic bonds, and have a good inhibitory effect on various pathogenic bacteria in plants, such as those causing wheat sharp eyespot and tobacco black shank. (3) Glucooligosaccharides: a series of oligosaccharide elicitors that people studied at first and knew systematically; can effectively induce the synthesis and accumulation of phytoalexins; serve as early informational molecules that are of great significance for plants in: the resistance to diseases and infection, molecular signal regulation, growth and development, morphogenesis, adaptation to the environment and other aspects; and have favorable prevention and treatment effects on wheat powdery mildew, potato late blight, etc. (4) Oligogalacturonic acids: oligosaccharides composed of 2-20 galacturonic acids linked by α-1,4-glycosidic bonds, derived from pectic polysaccharides and widely distributed in primary cell walls and intercellular layers of roots, stems, leaves and fruits of higher plants; play a role in softening and adhesion of cell tissues; and have favorable prevention and treatment effects on pepper virus and apple mosaic virus diseases.

One embodiment of the present disclosure provides a heteropoly oligosaccharide. The heteropoly oligosaccharide contains seven D-glucose residues and one D-galactose residue. Each heteropoly oligosaccharide molecule comprises R₁ and R₂, R₁ being H or a monomolecular pyruvate group, R₂ being H or a monomolecular succinyl group, and the structure thereof is represented as follows:

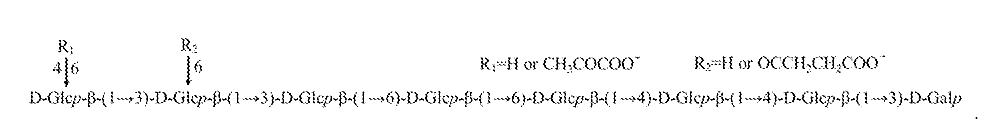

In one example, when R1 is pyruvate group and R2 is H, a chemical formula of the heteropoly oligosaccharide is as follows:

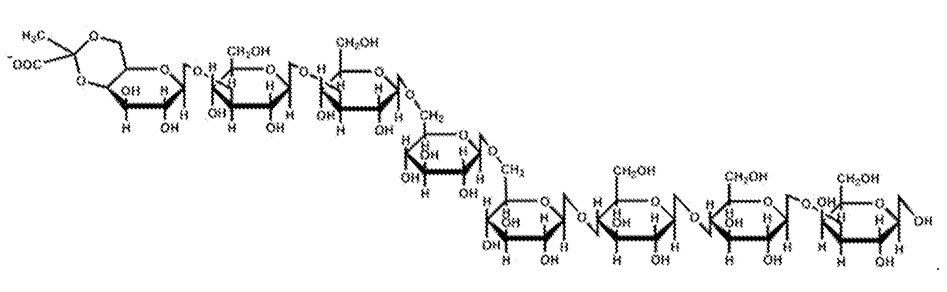

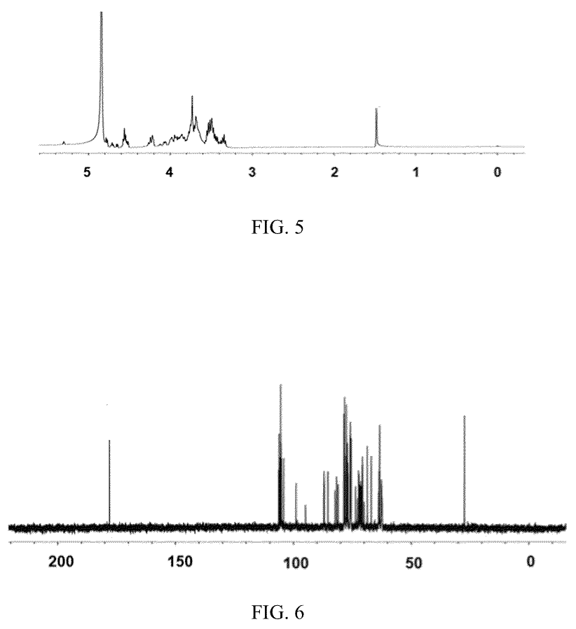

FIG. 5 shows hydrogen nuclear magnetic resonance (NMR) spectrum of the heteropoly oligosaccharide consistent with some embodiments of the present disclosure. FIG. 6 shows carbon-13 NMR spectrum of the heteropoly oligosaccharide consistent with some embodiments of the present disclosure. FIGS. 5-6 are plots of chemical shift in ppm (spectrum x-axis) versus signal intensity (spectrum y-axis). In the structure formula of the heteropoly oligosaccharide corresponding to the spectrum shown in FIGS. 5-6, R1 is a pyruvyl group, and R2 is H. Table 1 shows chemical shift of the heteropoly oligosaccharide in the ¹H and ¹³C NMR spectrum shown in FIGS. 5-6.

TABLE 1

chemical shift of heteropoly oligosaccharide in ¹H and ¹³C NMR spectrum

| sugar residue | nucleus | chemical shift (ppm, δ) | | | | | | |
|---|---|---|---|---|---|---|---|---|
| | | 1 | 2 | 3 | 4 | 5 | 6 | 6′ |
| α-D-Glap | ¹H | 5.30 | 4.00 | 3.99 | 4.24 | 4.12 | 3.75 | - |
| Aα | ¹³C | 94.83 | 70.66 | 82.38 | 71.94 | 72.97 | 64.11 | |
| β-D-Galp | ¹H | 4.66 | 3.66 | 3.81 | 4.20 | 3.72 | 3.78 | - |
| Aβ | ¹³C | 98.83 | 73.72 | 85.49 | 71.57 | 77.61 | 63.95 | |
| →1,4-β-D-Glcp | ¹H | 4.72 | 3.44 | 3.68 | 3.68 | 3.61 | 3.85 | - |
| B | ¹³C | 106.27 | 75.96 | 77.23 | 81.86 | 78.28 | 62.64 | |
| →1,4-β-D-Glcp | ¹H | 4.58 | 3.39 | 3.67 | 3.67 | 3.64 | 3.83 | 3.99 |
| C | ¹³C | 104.99 | 76.91 | 77.33 | 82.57 | 78.38 | 63.52 | |
| →1,6-β-D-Glcp | ¹H | 4.51 | 3.36 | 3.53 | 3.53 | 3.46 | 3.86 | 4.22 |
| D | ¹³C | 105.61 | 75.79 | 79.15 | 72.53 | 77.75 | 72.63 | |
| →1,6-β-D-Glcp | ¹H | 4.54 | 3.36 | 3.53 | 3.53 | 3.48 | 3.89 | 4.21 |
| E | ¹³C | 105.37 | 75.85 | 79.15 | 72.41 | 77.75 | 71.94 | |
| →1,3-β-D-Glcp | ¹H | 4.56 | 3.54 | 3.76 | 3.52 | 3.50 | 3.76 | 3.93 |
| F | ¹³C | 105.25 | 75.96 | 87.50 | 71.57 | 78.74 | 68.80 | |
| →1,3-β-D-Glcp | ¹H | 4.76 | 3.54 | 3.74 | 3.51 | 3.50 | 3.73 | 3.91 |
| G | ¹³C | 105.36 | 75.96 | 87.50 | 71.23 | 78.74 | 64.11 | |
| →1,4,6-β-D-Glcp | ¹H | 4.78 | 3.43 | 3.70 | 3.47 | 3.48 | 3.68 | 4.07 |
| H | ¹³C | 106.02 | 78.73 | 78.28 | 79.15 | 68.79 | 67.09 | |

The present disclosure also provides a preparation method for the heteropoly oligosaccharide. The heteropoly oligosaccharide is prepared through enzymolysis of an exopolysaccharide Riclin under an action of β-glucanase, the exopolysaccharide Riclin being produced from Agrobacterium sp. ZCC3656 (CCTCC No.: M 2018797). Here, CCTCC is abbreviation of China Center for Type Culture Collection.

In some embodiments, the preparation method may include the following steps.

### Enzymolysis of Polysaccharide Riclin

An exopolysaccharide Riclin produced from Agrobacterium sp. ZCC3656 (CCTCC No.: M 2018797) is dissolved in an aqueous solution, β-glucanase is added to obtain a mixture. The mixture is placed in a thermostatic water bath at 60° C. for reaction and vibrated until the exopolysaccharide Riclin is enzymolyzed completely.

### Purification of Heteropoly Oligosaccharide

A solution obtained from the enzymolysis in step (1) is centrifuged to remove insoluble impurities and obtain a supernatant. A mixed solvent of chloroform and n-butanol at a volume ratio of 4:1 is added to the supernatant to remove proteins from the supernatant. The solution is vibrated vigorously and left to stand for stratification, the aqueous phase is taken and centrifuged to remove the protein layer, and the supernatant is retained. These steps are repeated several times until the protein layer is removed completely. Finally, 95% ethanol is added to precipitate a heteropoly oligosaccharide.

The Agrobacterium sp. ZCC3656 has been disclosed in Chinese Patent Application No. 201811493131.2.

The present disclosure also provides an application of the heteropoly oligosaccharide in improving plant disease resistance.

In some embodiments, the plants include but are not limited to crops and other plants, such as tobacco, wheat, tomato, potato, apple, strawberry, paddy, and soybean.

In some embodiments, for the application of the heteropoly oligosaccharide in improving plant disease resistance, the plant disease resistance is the resistance to pathogenic bacteria infection in plants, including but not limited to the disease resistance to mosaic virus infection, disease resistance to Fusarium graminearum infection, disease resistance to Cladosporium ƒulvum infection, disease resistance to Phytophthora infestans infection, disease resistance to Marssonina mali infection and disease resistance to Pseudomonas solanacearum infection.

The present disclosure also provides a plant disease resistance inducer with an active ingredient containing the heteropoly oligosaccharide.

In the plant disease resistance inducer of the present disclosure, a concentration of the heteropoly oligosaccharide is 5-5,000 mg/L, and preferably 50-200 mg/L.

The heteropoly oligosaccharide of the present disclosure applied to crops and various other plants as a plant disease resistance inducer can significantly improve plant disease resistance, specifically as follows. 1) When acting on tobacco leaves, it can obviously increase the concentration of hydrogen peroxide in tobacco leaves as the dose increases; 2) When acting on lower epidermal cells of wheat leaves, it can obviously induce the release of hydrogen peroxide from cells of wheat leaves as the fluorescence intensity increases; 3) When acting on tomato leaves, it can obviously improve activities of glucanase, chitinase and phenylalanine ammonia lyase continuously as the dose increases and the induction time prolongs; 4) When acting on potato leaves infected with Phytophthora infestans patches, it can obviously reduce the damage of pathogenic bacteria to plants, and alleviate and even eliminate pathogenic bacteria infection.

Examples will be described below in detail in conjunction with the specific embodiments and accompanying drawings.

### Example 1: Screening and Purification of Heteropoly Oligosaccharide

### 1. Preparation and Separation of Polysaccharide

Agrobacterium sp. ZCC3656 was inoculated into an Htm (containing 1 g of sodium dihydrogen phosphate, 0.07 g of anhydrous calcium chloride, 0.2 g of magnesium chloride, 0.0125 g of ferrous sulfate, 3 g of potassium nitrate, 0.003 g of manganese sulfate, 0.0075 g of zinc chloride, 1,000 mL of water and 20 g of sucrose, with a pH value of 7.0-7.2) liquid medium, and cultured in a shaker at 28° C. and 250 rpm for 48 h; a 2-fold volume of industrial ethanol (95% ethanol) was added to the fermentation broth, and white filamentous polysaccharide precipitate was observed; the precipitate was collected through centrifugation at 6,000 × g and dried in a vacuum drying oven at 60° C. for 8 h, and the solid polysaccharide was pulverized with a grinder to obtain a crude polysaccharide.

### 2. Preparation of Heteropoly Oligosaccharide

The polysaccharide was weighed and dissolved in an aqueous solution, β-glucanase was added, the mixture was uniformly mixed and allowed for reaction in a thermostatic water bath at 60° C., and vibrated 1 h later until the polysaccharide solution became not viscous any more.

### 3. Purification of Heteropoly Oligosaccharide

The enzymolyzed solution was centrifuged at 9,000 rpm for 30 min to remove insoluble impurities. Proteins were removed from the centrifuged supernatant by a Sevage method, i.e., by adding a ¼ volume of a chloroform-n-butanol (4:1) mixture, the solution was vibrated vigorously for 30 min and left to stand for stratification, the aqueous phase was centrifuged at 7,000 rpm for 10 min to remove the protein layer, and the supernatant was transferred to a clean container. These operations were repeated several times until there was no protein layer. Finally, a 3-fold volume of 95% ethanol (e.g., the volume of 95% ethanol is three times of the volume of the result supernatant) was added to precipitate a heteropoly oligosaccharide, and the heteropoly oligosaccharide precipitate was dried in the vacuum drying oven at 50° C.

### Example 2: Application of Heteropoly Oligosaccharide in Improving Tobacco Mosaic Disease Resistance

### 1. Establishment of Tobacco Test Model

The test tobacco variety was Nicotiana X sanderae. The laboratory culture was conducted at a constant temperature of 30° C. for 12 h of lightness and 12 h of darkness, and the soil was kept moist. The test was divided into six groups: a control group sprayed with water, and groups sprayed with the heteropoly oligosaccharide at different concentrations (5 mg/L, 25 mg/L, 50 mg/L, 100 mg/L and 250 mg/L, respectively). The leaves were collected within 15 min after spraying to determine relevant signaling molecules.

### 2. Determination of H₂O₂ Concentration in Tobacco Leaves

100-200 mg of fresh plant leaves were weighed and quickly ground into superfine powder in liquid nitrogen; 1 mL of acetone was added, the mixture was extracted under vortex vibration and centrifuged at 10,000 rpm for 10 min, and the supernatant was transferred to a clean centrifuge tube; 0.8 mL of extract was taken and put in a 2 mL centrifuge tube, 0.1 mL of titanium tetrachloride reagent (5 mL of titanium tetrachloride reagent contains 2.175 mL of concentrated hydrochloric acid, 1 mL of titanium tetrachloride and 1.825 mL of deionized water) was added, then 0.2 mL of concentrated ammonia water was added, the solution was centrifuged at 5,000 rpm for 10 min, and the precipitate was collected; the precipitate was washed with acetone 3-5 times until the precipitate turned white; 1 mL of 2 M H₂SO₄ was added to the washed precipitate, and the precipitate was dissolved under vibration; 200 µL of sample was transferred to a 96-well colorimetric plate, and the absorbance value at 415 nm was read. The results are shown in FIG. 2.

### 3. Establishment of Tobacco Mosaic Disease Model

The test tobacco variety was Nicotiana X sanderae. The outdoor culture was conducted, and the soil was kept moist. The test was divided into five groups: a control group sprayed with water, and groups sprayed with the heteropoly oligosaccharide at different concentrations (50 mg/L, 100 mg/L, 200 mg/L and 500 mg/L, respectively). A tobacco mosaic virus was evenly sprayed on surfaces of tobacco leaves two days after the heteropoly oligosaccharide was sprayed, and the leaves were observed for infection. After the tobacco mosaic virus was sprayed, infection conditions of tobacco leaves were recorded every other week for three consecutive weeks. The results are shown in Table 2.

TABLE 2

Application of heteropoly oligosaccharide in improving tobacco mosaic disease resistance
Statistics of preventive test before onset of tobacco mosaic disease

| Concentration of heteropoly oligosaccharide (mg/L) | One week after spraying | | Two weeks after spraying | | Three weeks after spraying | |
|---|---|---|---|---|---|---|
| | Control effect (%) | Incidence (%) | Control effect (%) | Incidence (%) | Control effect (%) | Incidence (%) |
| 0 | | 10.47 | | 38.31 | | 68.32 |
| 50 | 50.56 | 2.51 | 62.58 | 11.83 | 81.36 | 12.73 |
| 100 | 62.37 | 1.86 | 75.44 | 6.39 | 84.59 | 10.53 |
| 200 | 60.31 | 1.31 | 79.39 | 4.72 | 90.21 | 6.69 |
| 500 | 67.62 | 0.89 | 82.54 | 3.39 | 94.86 | 3.51 |

### 4. Data Analysis

To compare different groups, statistical analysis was conducted by an ANOVA statistical method. P<0.05 was considered to indicate a significant difference.

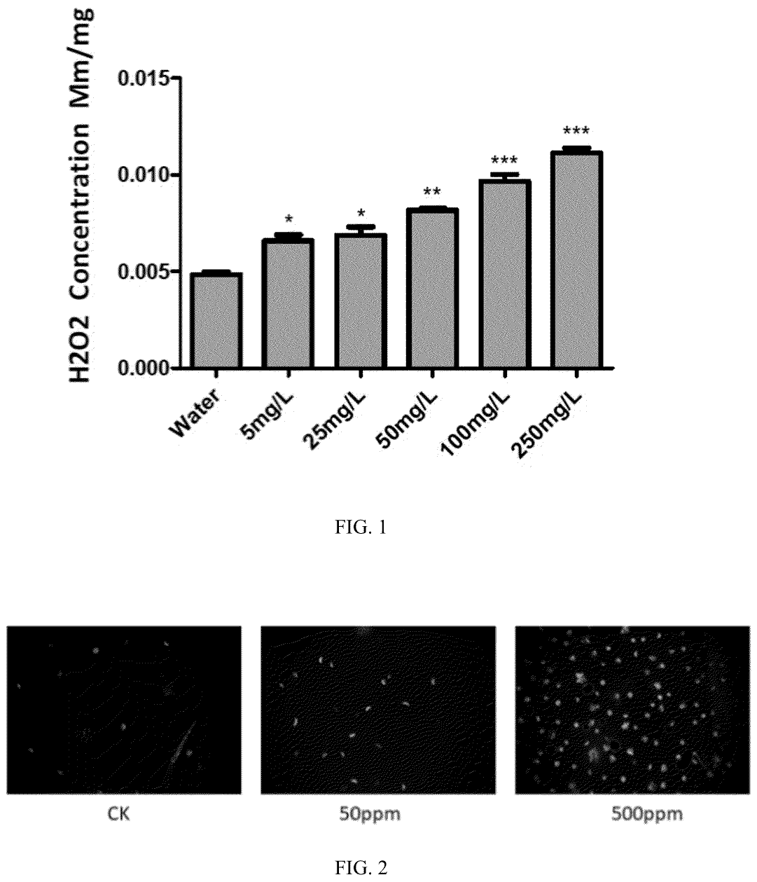

It can be seen from FIG. 1 and Table 2 that the concentration of hydrogen peroxide in tobacco leaves obviously increased after the heteropoly oligosaccharide was sprayed. The concentration of hydrogen peroxide increased linearly and dependently as the concentration of the heteropoly oligosaccharide increased. According to the mosaic virus infection conditions of tobacco, the heteropoly oligosaccharide can obviously improve the resistance of tobacco to mosaic virus, enhance prevention and treatment effects on tobacco against the virus, and reduce the infection rate of tobacco leaves. Thus, the heteropoly oligosaccharide can be a potential biological pesticide for improving plant disease resistance.

### Example 3: Application of Heteropoly Oligosaccharide in Improving Wheat Scab Resistance

### 1. Establishment of Wheat Test Model

The test wheat variety was Miannong No. 6. The laboratory culture was conducted at a constant temperature of 25° C. for 12 h of lightness and 12 h of darkness, and the soil was kept moist.

### 2. Fluorescence Detection of Hydrogen Peroxide in Wheat Leaves

The test was divided into three groups: a blank control group (i.e., using water as a control), a low-concentration heteropoly oligosaccharide group (with a final oligosaccharide concentration of 50 mg/L), and a high-concentration heteropoly oligosaccharide group (with a final oligosaccharide concentration of 500 mg/L). The fluorescence detection of H₂O₂ was conducted by the following method: H₂DCF-DA was prepared into a 1 mmol/L stock solution by DMSO, lower epidermis was carefully torn from plant leaves and incubated in a citric acid buffer with a pH value of 5.6 for 4 h, then the citric acid buffer was replaced, H₂DCF-DA was added until the final concentration reached 10 µmol/L, and incubation was conducted under horizontal shaking for 30 min; upon completion of incubation, excess fluorescent dye was removed through 3 times of rinsing with the citric acid buffer; the lower epidermis of leaves was placed on a glass slide, and the fluorescence intensity was observed using a BP460-490 laser color filter within a light wavelength range of 460-490 nm. The results are shown in FIG. 2.

### 3. Establishment of Wheat Scab Model

The test wheat variety was Miannong No. 6. The outdoor culture was conducted, and the soil was kept moist. The test was divided into five groups: a control group sprayed with water, and groups sprayed with the heteropoly oligosaccharide at different concentrations (50 mg/L, 100 mg/L, 200 mg/L and 500 mg/L, respectively). A Fusarium graminearum spore suspension was evenly sprayed on surfaces of wheat leaves two days after the heteropoly oligosaccharide was sprayed, and the leaves were observed for infection. Infection conditions of wheat leaves were recorded two weeks after the Fusarium graminearum spore suspension was sprayed. The results are shown in Table 3.

$$\begin{matrix}\text{Incidence} = \\ \text{number} \text{of} \text{diseased} \text{plants} / \text{number} \text{of} \text{investigated} \text{plants} \times \\ 100 \% ;\end{matrix}$$

$$\begin{matrix}\text{Disease} \text{index} = \sum \left(\text{number} \text{of} \text{diseased} \text{leaves} \text{at} \text{all} \text{levels}\right) \times \\ \left(\text{value} \text{of} \text{corresponding} \text{level}\right) / \left(\text{total} \text{number} \text{of} \text{investigated} \text{leaves} \times \right) \\ \left(\text{representative} \text{value} \text{of} \text{highest} \text{level}\right) \times 100 ;\end{matrix}$$

$$\begin{matrix}\text{Control} \text{effect} = \left(\text{disease} \text{index} \text{of} \text{control} \text{group} \text{-}\right) \\ \left(\text{disease} \text{index} \text{of} \text{treatment} \text{group}\right) / \text{disease} \text{index} \text{of} \text{control} \text{group} \times \\ 100 \% .\end{matrix}$$

TABLE 3

Application of heteropoly oligosaccharide in improving wheat scab resistance
Statistics of preventive test before onset of wheat scab

| Concentration of heteropoly oligosaccharide (mg/L) | Incidence (%) | Disease index | Control effect (%) |
|---|---|---|---|
| 0 | 32.1 | 40.6 | |
| 50 | 6.4 | 10.5 | 74.1 |
| 100 | 6.2 | 8.7 | 78.6 |
| 200 | 4.6 | 6.9 | 83.0 |
| 500 | 3.7 | 3.7 | 90.9 |

### 4. Data Analysis

To compare different groups, statistical analysis was conducted by an ANOVA statistical method. P<0.05 was considered to indicate a significant difference.

It can be seen from FIG. 2 and Table 3 that the fluorescence intensity of hydrogen peroxide produced by stomatal cells was low when lower epidermal cells of wheat leaves were not induced by the heteropoly oligosaccharide, and the heteropoly oligosaccharide with a final concentration of 500 mg/L can obviously induce stomatal cells to produce a large amount of hydrogen peroxide. According to the scab infection conditions of wheat, spraying the heteropoly oligosaccharide can improve the resistance of wheat to Fusarium graminearum spores. After the heteropoly oligosaccharide was sprayed, the control effect significantly increased and the incidence obviously decreased. Thus, the heteropoly oligosaccharide can be a potential biological pesticide for improving plant disease resistance.

### Example 4: Application of Heteropoly Oligosaccharide in Improving Tomato Leaf Mold Resistance

### 1. Establishment of Tomato Test Model

The test tomato variety was Huizhen No. 1. The laboratory culture was conducted at a constant temperature of 25° C. for 12 h of lightness and 12 h of darkness, and the soil was kept moist.

### 2. Activity Determination of Enzymes Related to Tomato Leaf Resistance

The test was divided into three groups: a blank control group (i.e., using water as a control), a low-concentration heteropoly oligosaccharide group (with a final oligosaccharide concentration of 50 mg/L), and a high-concentration heteropoly oligosaccharide group (with a final oligosaccharide concentration of 150 mg/L). Samples were taken 4 h, 24 h and 72 h after the heteropoly oligosaccharide was sprayed to determine activities of relevant proteases.

(1) Extraction of crude enzyme: 25 mg of leaves were quickly ground into superfine powder in liquid nitrogen, and 500 µL of protein extract (containing a 20 mM PBS buffer, 5 mM EDTA2·Na, 2.5 mM DTT, 2.5 mM Na₂S₂O₃·5H₂Oand 0.5% pvp) was added.

(2) Activity determination of glucanase: A reaction solution contained 200 µL of 1% laminarin, 100 µL of crude enzyme extract, and 200 µL of 50 mM sodium acetate buffer with a pH value of 5.0 which were mixed uniformly, the mixture was allowed for reaction at 37° C. for 2 h and then put in a boiling water bath for 10 min, and the reaction was terminated; 100 µL of enzymolysis solution was taken, 100 µL of DNS was added, the resulting solution was treated in the boiling water bath for 5 min and then cooled in an ice water bath, and the absorbance value at 540 nm was read.

(3) Activity determination of chitinase: A reaction solution contained 200 µL of chitin substrate, 100 µL of crude enzyme extract, and 200 µL of 50 mM sodium acetate buffer with a pH value of 5.0 which were mixed uniformly, the mixture was allowed for reaction at 37° C. for 2 h and then put in a boiling water bath for 10 min, and the reaction was terminated; 100 µL of enzymolysis solution was taken, 100 µL of DNS was added, the resulting solution was treated in the boiling water bath for 5 min and then cooled in an ice water bath, and the absorbance value at 540 nm was read.

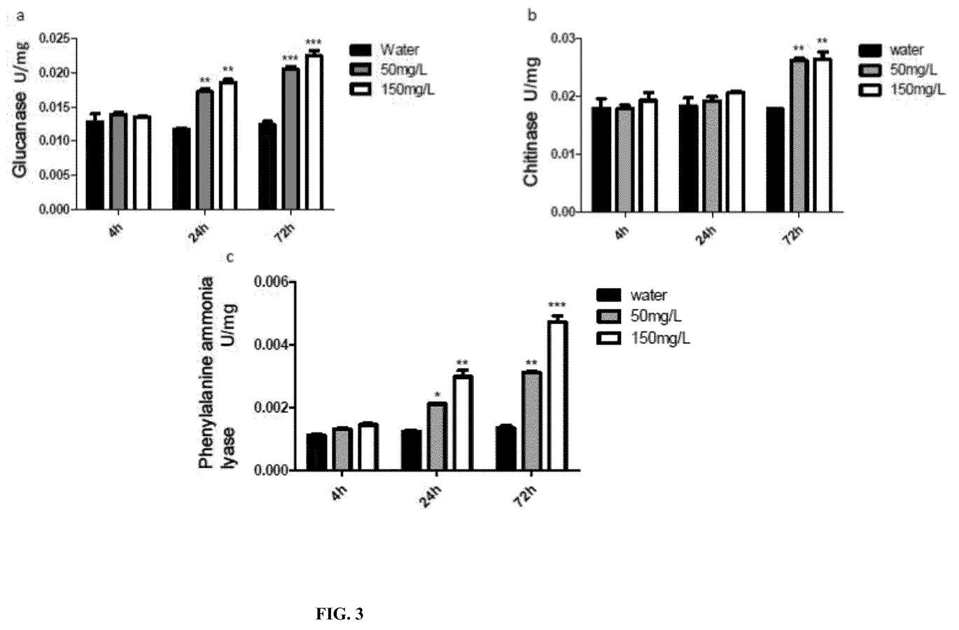

(4) Activity determination of phenylalanine ammonia lyase: A reaction solution contained 100 µL of crude enzyme extract, 500 µL of 20 mM L-phenylalanine solution, and 400 µL of 50 mM Tris-HCl buffer with a pH value of 8.5, and reacted at 37° C. for 2 h; 0.2 mL of 6 M HCl was added to terminate the reaction, 2 mL of ether was added to extract cinnamic acid, and the solution was centrifuged at 3,000 rpm for 1 min; the extractant was transferred to a clean 5 mL centrifuge tube, the extraction was repeated once, and extracts were put together and dried with nitrogen; 0.5 mL of methanol was added for re-dissolution, and the absorbance value at 290 nm was read. The results are shown in FIG. 3.

### 3. Establishment of Tomato Leaf Mold Model

The test tomato variety was Huizhen No. 1. The outdoor culture was conducted, and the soil was kept moist. The test was divided into five groups: a control group sprayed with water, and groups sprayed with the heteropoly oligosaccharide at different concentrations (50 mg/L, 100 mg/L, 200 mg/L and 500 mg/L, respectively). A Cladosporium fulvum spore suspension was evenly sprayed on surfaces of tomato leaves two days after the heteropoly oligosaccharide was sprayed, and the leaves were observed for infection. Infection conditions of tomato leaves were recorded two weeks after the Cladosporium ƒulvum spore suspension was sprayed. The results are shown in Table 4.

$$\begin{matrix}\text{Incidence} = \\ \text{number} \text{of} \text{diseased} \text{plants} / \text{number} \text{of} \text{investigated} \text{plants} \times \\ 100 \% ;\end{matrix}$$

$$\begin{matrix}\text{Disease} \text{index} = \sum \left(\text{number} \text{of} \text{diseased} \text{leaves} \text{at} \text{all} \text{levels}\right) \times \\ \left(\text{value} \text{of} \text{corresponding} \text{level}\right) / \left(\text{total} \text{number} \text{of} \text{investigated} \text{leaves} \times \right) \\ \left(\text{representative} \text{value} \text{of} \text{highest} \text{level}\right) \times 100 ;\end{matrix}$$

$$\begin{matrix}\text{Control} \text{effect} = \left(\text{disease} \text{index} \text{of} \text{control} \text{group} \text{-}\right) \\ \left(\text{disease} \text{index} \text{of} \text{treatment} \text{group}\right) / \text{disease} \text{index} \text{of} \text{control} \text{group} \times \\ 100 \% .\end{matrix}$$

TABLE 4

Application of heteropoly oligosaccharide in improving tomato leaf mold resistance
Statistics of preventive test before onset of tomato leaf mold

| Concentration of heteropoly oligosaccharide (mg/L) | Incidence (%) | Disease index | Control effect (%) |
|---|---|---|---|
| 0 | 42.6 | 58.7 | |
| 50 | 8.5 | 12.6 | 78.5 |
| 100 | 7.2 | 11.9 | 79.7 |
| 200 | 5.7 | 8.8 | 85.0 |
| 500 | 2.3 | 5.7 | 90.3 |

### 4. Data Analysis

To compare different groups, statistical analysis was conducted by an ANOVA statistical method. P<0.05 was considered to indicate a significant difference.

It can be seen from FIG. 3 and Table 4 that after the heteropoly oligosaccharide was sprayed onto tomato leaves, the activity of chitinase significantly increased at 72 h, while activities of glucanase and phenylalanine ammonia lyase significantly increased at 24 h. Spraying the heteropoly oligosaccharide to tomato can improve the resistance of tomato to Cladosporium ƒulvum spores. After the heteropoly oligosaccharide was sprayed, the control effect significantly increased and the incidence obviously decreased. Thus, the heteropoly oligosaccharide can be a potential biological pesticide for improving plant disease resistance.

### Example 5: Application of Heteropoly Oligosaccharide in Improving Potato Phytophthora Disease Resistance

### 1. Establishment of Potato Test Model

The test potato variety was Fovorita. The laboratory culture was conducted at a constant temperature of 25° C. for 12 h of lightness and 12 h of darkness, and the soil was kept moist.

### 2. Test of Potato Phytophthora Infestans Infection

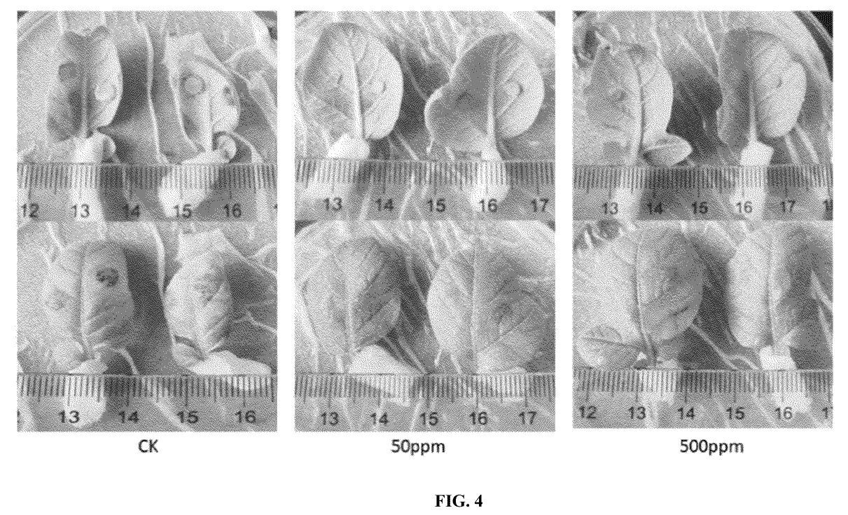

The test was divided into three groups: a blank control group (i.e., using water as a control), a low-concentration heteropoly oligosaccharide group (with a final oligosaccharide concentration of 50 mg/L), and a high-concentration heteropoly oligosaccharide group (with a final oligosaccharide concentration of 500 mg/L). Before the test, potato leaves were placed on a flat plate, and petioles were kept wet to prevent leaves from withering. The leaves were sprayed with different concentrations of the heteropoly oligosaccharide, kept wet and cultured at 20° C. for 36 h. Phytophthora infestans patches were attached onto potato leaves. The leaves were kept away from light for 24 h, and cultured for 12 h of lightness and 12 h of darkness. Two days later, infection conditions of leaves with patches were observed. The results are shown in FIG. 4.

### 3. Field Test of Potato Phytophthora Infestans Infection

The test was divided into four groups: a blank control group (i.e., using water as a control), a low-concentration heteropoly oligosaccharide group (with a final oligosaccharide concentration of 50 mg/L), a medium-concentration heteropoly oligosaccharide group (with a final oligosaccharide concentration of 250 mg/L), and a high-concentration heteropoly oligosaccharide group (with a final oligosaccharide concentration of 500 mg/L). After a Phytophthora infestans spore suspension was sprayed, infection conditions of potato leaves were recorded every other week for three consecutive weeks.

### 4. Data Analysis

It can be seen from FIG. 4 and Table 5 that after the heteropoly oligosaccharide was sprayed onto potato leaves, under the condition of infection with pathogenic bacteria patches, severe leaf infection was observed from the control group not treated with the heteropoly oligosaccharide, while leaves sprayed with the heteropoly oligosaccharide were slightly infected or even not infected. Spraying the heteropoly oligosaccharide to potato can improve the resistance of potato to Phytophthora infestans infection. After the heteropoly oligosaccharide was sprayed, the control effect significantly increased and the incidence significantly decreased. Thus, the oligosaccharide can favorably induce potato leaves to produce resistance and improve disease resistance, and can be a potential biological pesticide.

TABLE 5

Application of heteropoly oligosaccharide in improving potato late blight resistance
Statistics of preventive test before onset of potato late blight

| Concentration of heteropoly oligosaccharide (mg/L) | One week after spraying | | Two weeks after spraying | | Three weeks after spraying | |
|---|---|---|---|---|---|---|
| | Control effect (%) | Incidence (%) | Control effect (%) | Incidence (%) | Control effect (%) | Incidence (%) |
| 0 | | 26.53 | | 42.31 | | 72.32 |
| 50 | 60.21 | 4.54 | 79.42 | 8.71 | 87.54 | 12.51 |
| 250 | 63.56 | 3.32 | 82.67 | 7.33 | 90.21 | 10.69 |
| 500 | 70.39 | 0.82 | 87.39 | 5.34 | 95.36 | 7.17 |

### Example 6: Application of Heteropoly Oligosaccharide in Improving Apple Brown Spot Resistance

### 1. Establishment of Apple Test Model

The test apple variety was Red Fuji. The outdoor culture was conducted, and the soil was kept moist. The test was performed during a lush growth of leaves and before apples were borne.

### 2. Test of Marssonina Mali Infection

The test was divided into three groups: a blank control group (i.e., using water as a control), a low-concentration heteropoly oligosaccharide group (with a final oligosaccharide concentration of 50 mg/L), and a high-concentration heteropoly oligosaccharide group (with a final oligosaccharide concentration of 500 mg/L). A Marssonina mali spore suspension was evenly sprayed on surfaces of apple tree leaves two days after the heteropoly oligosaccharide was sprayed, and the leaves were observed for infection. After the Marssonina mali spore suspension was sprayed, infection conditions of apple tree leaves were recorded every other week for three consecutive weeks. The results are shown in Table 6.

TABLE 6

Application of heteropoly oligosaccharide in improving apple brown spot resistance
Statistics of preventive test before onset of apple brown spot

| Concentration of heteropoly oligosaccharide (mg/L) | One week after spraying | | Two weeks after spraying | | Three weeks after spraying | |
|---|---|---|---|---|---|---|
| | Control effect (%) | Incidence (%) | Control effect (%) | Incidence (%) | Control effect (%) | Incidence (%) |
| 0 | | 31.34 | | 50.13 | | 74.02 |
| 50 | 68.54 | 7.12 | 74.87 | 12.60 | 84.79 | 14.65 |
| 500 | 76.77 | 4.21 | 85.32 | 7.36 | 93.21 | 8.75 |

### 3. Data Analysis

It can be seen from Table 6 that after the heteropoly oligosaccharide was sprayed onto apple tree leaves, under the condition of infection with the pathogenic bacteria spore suspension, severe leaf infection was observed from the control group not treated with the heteropoly oligosaccharide, while leaves sprayed with the heteropoly oligosaccharide were slightly or even not infected. Spraying the heteropoly oligosaccharide to apple trees can improve the resistance of apple trees to Marssonina mali. After the heteropoly oligosaccharide was sprayed, the incidence obviously decreased. Thus, the heteropoly oligosaccharide can be a potential biological pesticide for improving plant disease resistance.

### Example 7: Application of Heteropoly Oligosaccharide in Improving Strawberry Bacterial Wilt Resistance

### 1. Establishment of Strawberry Test Model

The test strawberry variety was Ningxin. The outdoor culture was conducted, and the soil was kept moist. The test was performed during a lush growth of leaves and before strawberries were borne.

### 2. Test of Strawberry Pseudomonas Solanacearum Infection

The test was divided into five groups: a control group sprayed with water, and groups sprayed with the heteropoly oligosaccharide at different concentrations (50 mg/L, 100 mg/L, 200 mg/L and 500 mg/L, respectively). A strawberry Pseudomonas solanacearum suspension was evenly sprayed on surfaces of strawberry leaves two days after the heteropoly oligosaccharide was sprayed, and the leaves were observed for infection. Infection conditions of strawberry leaves were recorded two weeks after the Pseudomonas solanacearum suspension was sprayed. The results are shown in Table 7.

$$\begin{matrix}\text{Incidence} = \\ \text{number} \text{of} \text{diseased} \text{plants} / \text{number} \text{of} \text{investigated} \text{plants} \times 100 \% ;\end{matrix}$$

$$\begin{matrix}\text{Disease} \text{index} = \sum \left(\text{number} \text{of} \text{diseased} \text{leaves} \text{at} \text{all} \text{levels}\right) \times \\ \left(\text{value} \text{of} \text{corresponding} \text{level}\right) / \left(\text{total} \text{number} \text{of} \text{investigated} \text{leaves} \times \right) \\ \left(\text{representative} \text{value} \text{of} \text{highest} \text{level}\right) \times 100 ;\end{matrix}$$

$$\begin{matrix}\text{Control} \text{effect} = \left(\text{disease} \text{index} \text{of} \text{control} \text{group} \text{-}\right) \\ \left(\text{disease} \text{index} \text{of} \text{treatment} \text{group}\right) / \text{disease} \text{index} \text{of} \text{control} \text{group} \times \\ 100 \% .\end{matrix}$$

TABLE 7

Application of heteropoly oligosaccharide in improving strawberry bacterial wilt resistance
Statistics of preventive test before onset of strawberry bacterial wilt

| Concentration of heteropoly oligosaccharide (mg/L) | Incidence (%) | Disease index | Control effect (%) |
|---|---|---|---|
| 0 | 37.8 | 58.7 | |
| 50 | 6.8 | 14.4 | 75.5 |
| 100 | 5.2 | 10.7 | 81.8 |
| 200 | 5.1 | 10.2 | 82.6 |
| 500 | 2.3 | 6.6 | 88.8 |

### 3. Data Analysis

It can be seen from Table 7 that after the heteropoly oligosaccharide was sprayed onto strawberry leaves, under the condition of infection with the Pseudomonas solanacearum suspension, severe leaf infection was observed from the control group not treated with the heteropoly oligosaccharide, while leaves sprayed with the heteropoly oligosaccharide were slightly or even not infected. Spraying the heteropoly oligosaccharide to strawberries can improve the resistance of strawberries to Pseudomonas solanacearum. After the heteropoly oligosaccharide was sprayed, the incidence obviously decreased. Thus, the heteropoly oligosaccharide can be a potential biological pesticide for improving plant disease resistance.

Compared with the prior art, the present disclosure has the following advantages.

(1) The heteropoly oligosaccharide of the present disclosure can induce plants to produce the resistance to pathogenic bacteria infection with a significant induction effect, thereby obviously reducing occurrences of pathogenic bacteria infection in plants.

(2) A method of externally applying the heteropoly oligosaccharide is adopted in the present disclosure; the application method is simple, no biological toxicity is produced and no environmental pollution is caused.

(3) The heteropoly oligosaccharide of the present disclosure is prepared through strain fermentation by a simple method at a low cost, can be put into mass production and has a uniform structure, thereby having extensive application prospects as a new biological pesticide.

Those skilled in the art should understand that the modifications and variations of the various embodiments disclosed above can be made without departing from the essence of the invention. Therefore, the protection scope of the present disclosure should be defined by the appended claims.

Although different exemplary embodiments of the present disclosure have been described, it is obvious to those skilled in the art that various changes and modifications can be made, which can achieve some of the advantages of the present disclosure without departing from the spirit and scope of the present disclosure. For those who are quite skilled in the art, other components performing the same function can be replaced as appropriate. It should be mentioned that the features explained here with reference to particular figures can be combined with features of other figures, even in those cases where this is not explicitly mentioned. In addition, the method of the present disclosure can be implemented either in all software implementations using appropriate processor instructions or in a hybrid implementation using a combination of hardware logic and software logic to achieve the same result. Such modifications to the solution according to the invention are intended to be covered by the appended claims.

What is claimed is:

1. A heteropoly oligosaccharide, comprising:
seven D-glucose residues and one D-galactose residue,
wherein each heteropoly oligosaccharide molecule comprises R₁ and R₂, R₁ being H or a monomolecular pyruvate group, R₂ being H or a monomolecular succinyl group, and a structure of the heteropoly oligosaccharide molecule is represented as follows: 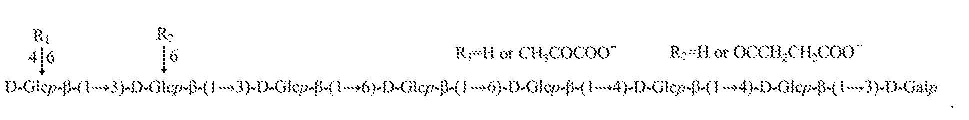 .

2. A preparation method for a heteropoly oligosaccharide, comprising:
producing an exopolysaccharide Riclin from Agrobacterium sp. ZCC3656 (CCTCC No.: M 2018797); and
preparing the heteropoly oligosaccharide through enzymolysis of the exopolysaccharide Riclin under an action of β-glucanase, wherein:
the heteropoly oligosaccharide comprises seven D-glucose residues and one D-galactose residue; and
each heteropoly oligosaccharide molecule comprises R₁ and R₂, R₁ being H or a monomolecular pyruvate group, R₂ being H or a monomolecular succinyl group, and a structure of the heteropoly oligosaccharide molecule is represented as follows: 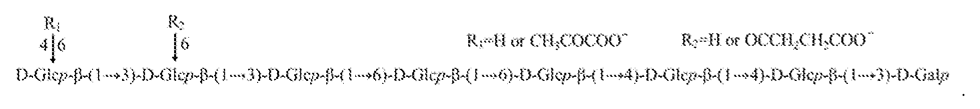 .

3. The preparation method according to claim 2, wherein:
the enzymolysis of the exopolysaccharide Riclin comprises:
dissolving the exopolysaccharide Riclin in an aqueous solution, and adding β-glucanase to obtain a mixture; and
placing the mixture in a thermostatic water bath at 60° C. for a reaction and vibrating until the exopolysaccharide Riclin is enzymolyzed completely.

4. The preparation method according to claim 3, further comprising purifying the heteropoly oligosaccharide, comprising:
centrifuging a solution obtained from the enzymolysis of the exopolysaccharide Riclin to remove insoluble impurities and obtain a supernatant;
adding, to the supernatant, a mixed solvent of chloroform and n-butanol at a volume ratio of 4:1 to remove proteins from the supernatant, vibrating vigorously and leaving to stand for stratification, taking an aqueous phase for centrifugation to remove a protein layer, retaining a result supernatant;
repeating the previous step multiple times until the protein layer is removed completely; and
adding 95% ethanol to precipitate the heteropoly oligosaccharide.

5. A plant disease resistance inducer, wherein an active ingredient of the plant disease resistance inducer to improve disease resistance of a plant comprises a heteropoly oligosaccharide, wherein:
the heteropoly oligosaccharide comprises seven D-glucose residues and one D-galactose residue; and
each heteropoly oligosaccharide molecule comprises R₁ and R₂, R₁ being H or a monomolecular pyruvate group, R₂ being H or a monomolecular succinyl group, and a structure of the heteropoly oligosaccharide molecule is represented as follows: 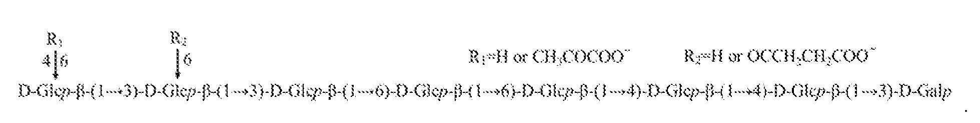 .

6. The plant disease resistance inducer according to claim 5, wherein the plant is tobacco, wheat, tomato, potato, apple, strawberry, paddy or soybean.

7. The plant disease resistance inducer according to claim 5, wherein the disease resistance of the plant is resistance to pathogenic bacteria infection in the plant.

8. The plant disease resistance inducer according to claim 5, wherein the disease resistance of the plant is disease resistance to mosaic virus infection, disease resistance to Fusarium graminearum infection, disease resistance to Cladosporium fulvum infection, disease resistance to Phytophthora infestans infection, disease resistance to Marssonina mali infection, or disease resistance to Pseudomonas solanacearum infection.

9. The plant disease resistance inducer according to claim 5, wherein a concentration of the heteropoly oligosaccharide is 5-5,000 mg/L.

10. The plant disease resistance inducer according to claim 5, wherein a concentration of the heteropoly oligosaccharide is 50-200 mg/L.
